(19) United States
(12) Patent Application Publication
(10) Pub. No.: US 2023/0069693 A1
(43) Pub. Date: Mar. 2, 2023

(54) TRAUMA-INTERVENTION DETERMINATION

(71) Applicants: Regents of the University of Minnesota, Minneapolis, MN (US); The Medical College of Wisconsin, Inc., Milwaukee, WI (US)

(72) Inventors: Christopher Tignanelli, Edina, MN (US); Benjamin Chen, Shoreview, MN (US); Rachel Morris, Fox Point, WI (US)

(21) Appl. No.: 17/817,561

(22) Filed: Aug. 4, 2022

Related U.S. Application Data
(60) Provisional application No. 63/260,123, filed on Aug. 10, 2021.

(51) Int. Cl. G16H 50/20

(52) U.S. Cl. CPC G16H 50/20

(57) ABSTRACT

A computer-based, trauma-patient-triage system includes one or more computing devices configured to: receive, from a mobile computing device, user input comprising a plurality of parameters indicating a condition of a trauma patient; apply the plurality of parameters to one or more machine-learning algorithms trained to determine, based on the plurality of parameters, a trauma-triage category for the patient, wherein the trauma-triage category for the patient indicates an NEI-6 designation for the patient; and transmit the trauma-triage category to the mobile computing device for display on the mobile computing device.

This application claims the benefit of U.S. Provisional Patent Application No. 63/260,123, filed Aug. 10, 2021, the entire contents of which is incorporated herein by reference.

### TECHNICAL FIELD

The disclosure relates to medical computing systems, and in particular, computing systems configured to triage trauma intervention.

### BACKGROUND

Annually, over 192,000 people die, and more than 3 million are hospitalized, due to injury in the United States. In 2013, injury was the leading cause of death among individuals under the age of 45 years and had an estimated economic burden of $671 billion. It has been well-established that regional trauma systems have been fundamental in reducing the mortality associated with injury. The subject of trauma triage may be divided into 3 subcategories: “field-triage” (e.g., choice of destination hospital), “hospital-triage” (e.g., level of trauma-team activation) and “triage-assessment” (e.g., assessment of the appropriateness of trauma-team activation as it relates to injury severity).

### SUMMARY

In general, this disclosure describes computer-implemented systems and techniques for rapidly and accurately determining (or “predicting”) an appropriate trauma-triage level for a trauma patient, based on prehospital metrics available to first responders in the prehospital setting. For example, this disclosure describes one or more machine-learning (ML), artificial-intelligence (AI) or deep-learning (DL) based models configured to determine a Need for Emergent Intervention within 6 hours (“NEI-6”) designation for a trauma patient, based on a plurality of parameters indicative of the patient's condition. In some examples, a machine-learning-based NEI-6-designation predictive model may be implemented in a mobile-computing application configured to designate the patient as either “NEI-6 Positive,” indicating that a full trauma-team activation (TTA) is likely required for the patient, or instead, as “NEI-6 Negative,” such that a first responder, such as EMS personnel, can appropriately triage the trauma patient.

In this way, available medical resources may be significantly more-accurately and more-efficiently allotted according to the needs of patients, as compared to other triage procedures, e.g., based only on a patient's Injury Severity Score (ISS). For instance, the more-accurate patient-triage techniques described herein may significantly reduce instances of wasted medical resources due to patient-overtriage, as well as significantly reduce preventable instances of patient mortality due to patient-undertriage.

In some examples, a computing system includes processing circuitry configured to: receive, from a mobile computing device, user input comprising a plurality of parameters indicating a condition of a trauma patient; apply the plurality of parameters to one or more machine-learning algorithms trained to determine, based on the plurality of parameters, a trauma-triage category for the patient, wherein the trauma-triage category for the patient indicates a trauma-triage recommendation for the patient; and transmit the trauma-triage category to the mobile computing device for display on the mobile computing device.

In some examples, a computing device includes processing circuitry configured to: receive, via a user interface, user input comprising a plurality of parameters indicating a condition of a trauma patient; transmit the plurality of parameters to a remote server configured to apply the plurality of parameters to one or more machine-learning algorithms trained to determine, based on the plurality of parameters, a trauma-triage category for the patient, wherein the trauma-triage category for the patient indicates a trauma-triage recommendation for the patient; receive the trauma-triage category for the patient from the remote server; and output the trauma-triage category for the patient for display via the user interface.

In some examples, a method includes: receiving, by first processing circuitry of a mobile computing device via a user interface of the mobile computing device, user input comprising a plurality of parameters indicating a condition of a trauma patient; wirelessly transmitting, by the first processing circuitry, the plurality of parameters to a remote server; receiving, by second processing circuitry of the remote server, the plurality of parameters; applying, by the second processing circuitry, the plurality of parameters to one or more machine-learning algorithms trained to determine, based on the plurality of parameters, a trauma-triage category for the patient, wherein the trauma-triage category for the patient indicates a trauma-triage recommendation for the patient; wirelessly transmitting, by the second processing circuitry to the mobile computing device, the trauma-triage category for the patient; receiving, by the first processing circuitry from the remote server, the trauma-triage category; and outputting for display, by the first processing circuitry via the user interface, an indication of the trauma-triage category.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual block diagram illustrating an example computing system for determining appropriate triage levels for trauma patients.

FIG. 2 is a conceptual block diagram illustrating various example computing devices of the computing system of FIG. 1.

FIG. 3 is a bar graph illustrating Shapley values for an example validation dataset that may be used to evaluate the predictive models described herein.

FIG. 4 is a line graph depicting an example Area-Under-the-Receiving-Operator-Characteristic (“AUC” or “AUROC”) indicating the relative performance of the predictive models of this disclosure.

FIGS. 5A, 5B, and 5C are example GUIs of a mobile application implementing an NEI-6 predictive model, in accordance with techniques of this disclosure.

FIG. 6 is a flowchart illustrating an example operation in accordance with the present techniques.

### DETAILED DESCRIPTION

Trauma is the leading cause of death in the United States of America (US) and worldwide for people under the age of 45. Unfortunately, there are approximately 6 million traumatic deaths a year. This is significant, as more people die each year from trauma than from malaria, tuberculosis, and HIV/AIDs combined. Additionally, studies have found that nearly 20% of trauma deaths are preventable, with the majority of preventable deaths occurring in the prehospital setting. These deaths may be due to quality gaps in prehospital and intrahospital trauma systems. It has been well-established that regional trauma systems have been fundamental in reducing the mortality associated with injury.

Emergency medical services (EMS) workers are the first-line providers in the event of acute trauma. They play an essential role in ensuring that severely injured patients receive care at centers that can appropriately and effectively manage the patients' conditions. The subject of trauma triage may be divided into 3 subcategories: “field-triage” (e.g., choice of destination hospital), “hospital-triage” (e.g., level of trauma-team activation) and “triage-assessment” (e.g., assessment of the appropriateness of trauma-team activation as it relates to injury severity). Appropriate-hospital triage improves patient outcomes, prevents both the underutilization and the overutilization of dedicated trauma centers, and decreases unnecessarily incurred financial burdens.

Accurately matching the severity of a trauma patient's injury with an appropriate level of hospital triage to provide adequate resources remains a challenge. “Undertriage” occurs when a patient with severe injuries requires a full trauma-team activation (TTA) does not receive one, whereas “overtriage” occurs when a patient that does not require a TTA receives one, e.g., based on the patient's Injury Severity Score (ISS). The American College of Surgeons' Committee on Trauma (ACS-COT) has set undertriage and overtriage benchmarks at 5% and 35% respectively, yet undertriage rates of 24%-69% and overtriage rates of about 50% have been reported. Undertriage of severely injured patients is associated with increased patient-mortality rates. Overtriage places unnecessary financial burdens on patients, and can cause occupation “burnout” for medical providers.

A plausible explanation for these high undertriage rates may be multifactorial. First, existing trauma-hospital-triage procedures are often based only on the patient's Injury Severity Score (ISS). For instance, a patient ISS above or below certain threshold values may be used to determine a triage level for the patient. As one example, the current definition for “major trauma,” as utilized by the majority of hospital-triage models, is “an ISS greater than 15.”

However, the ISS model is highly simplistic, in that it depends only on patient data that includes less than ten percent of all causes of in-hospital mortality, resulting in high rates of both overtriage and undertriage. This simplistic definition does not enable optimal identification of severe injuries. Additionally, actual compliance rates with established hospital-triage protocols have been observed to vary widely. Finally, a majority of existing hospital-triage models do not adequately identify severely injured patients, primarily due to their relatively low sensitivities to certain significant determining factors.

Given the many limitations of the current ISS model, several other intervention-based definitions for major trauma have been proposed to measure TTA appropriateness, such as the “Need For Trauma Intervention” (“NFTI”), the “Secondary Triage Assessment Tool” (“STAT”), and the “Need for an Emergent Intervention within 6 hours” (“NEI-6”). It has previously been shown that NEI-6 performs better than currently available metrics in determining TTA appropriateness, in terms of undertriage, mortality, and need for resource utilization. An NEI-6 designation (NEI-6 Positive or NEI-6 Negative) is currently determined (e.g., defined) based on the presence of the following factors: (1) receiving ≥5 units of packed red blood cells within the first 4 hours, (2) any operation, (3) angiography, (4) chest tube placement, (5) central line placement, or (6) brain intervention (e.g., placement of an intracranial pressure monitor, craniotomy, etc.) within 6 hours of arrival.

Among available metrics, NEI-6 positive patients have the highest risk of undertriage death. The NEI-6 method may be associated with the lowest rate of undertriage. Establishing a validated model that accurately determines TTA-appropriateness by utilizing NEI-6 as a definition of major trauma could potentially lower both undertriage and overtriage rates observed for current protocols while maintaining similar or even decreased mortality rates. For instance, utilizing field variables to inform a predictive model can allow accurate hospital triage to occur in the prehospital setting, thereby ensuring an appropriate level of medical resources for a patient prior to their arrival. Accordingly, a predictive model for hospital triage, as described herein, may utilize a select combination of prehospital physiologic, anatomic, and mechanistic criteria available to an EMS provider at the scene, in order to accurately determine TTA appropriateness.

In general, this disclosure describes computer-implemented systems and techniques for rapidly and accurately determining (or “predicting”) an appropriate trauma-triage level for a trauma patient, based on prehospital metrics available to first responders. In particular, this disclosure describes one or more machine-learning (ML), artificial-intelligence (AI) or deep-learning (DL) based models configured to determine an NEI-6 designation for a trauma patient, based on a plurality of parameters indicative of the patient and the patient's condition. For instance, in some examples, a machine-learning model may be implemented in a mobile-computing application configured to designate the patient as “NEI-6 Positive,” indicating that a full trauma-team activation (TTA) is likely required for the patient, or instead, as “NEI-6 Negative,” such that a first responder, such as EMS personnel, can appropriately triage the trauma patient.

In this way, available medical resources may be significantly more-accurately and more-efficiently allotted according to the needs of patients, as compared to other triage procedures, e.g., based only on a patient's Injury Severity Score (ISS). For instance, the more-accurate patient-triage techniques described herein may significantly reduce instances of wasted medical resources due to patient-overtriage, as well as significantly reduce preventable instances of patient mortality due to patient-undertriage.

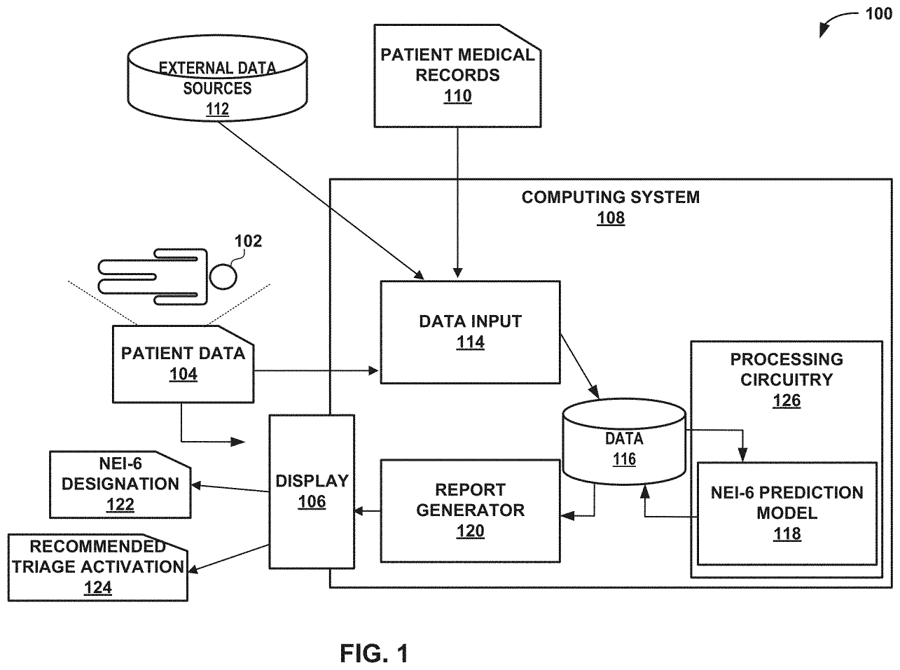

FIG. 1 is a block diagram illustrating an example NEI-6 determination system 100 for determining an NEI-6 designation 122, and in some examples, for determining a corresponding triage activation level 124, for a trauma patient 102. In general, NEI-6 determination system 100 incorporates criteria (e.g., patient data 104) available to an EMS provider in the prehospital setting to evaluate and determine a trauma patient's relative need for a TTA.

More specifically, system 100, as described herein, is the first robust trauma-triage system configured to use an “NEI-6” designation 122 for a patient 102 as the operative definition of “major trauma,” or in other words, as an objective determining factor as a basis for a corresponding triage-level recommendation 124. In this way, system 100 can more-accurately identify a trauma patient 102 who requires the greatest hospital resources (e.g., a full TTA), thereby significantly reducing undertriage-based patient mortality.

NEI-6 determination system 100 includes a computing system 108 having one or more computing devices, such as a mobile computing device (e.g., a smartphone, a tablet computer, a personal digital assistant, and the like), a desktop computing device, a server system, a distributed computing system (e.g., a “cloud” computing system), and/or any other device capable of receiving patient data 104 and performing the techniques described herein.

In examples described herein, computing system 108 includes processing circuitry 126 (e.g., one or more processors) configured to execute at least one artificial intelligence (AI), deep-learning (DL), or machine-learning (ML)-based NEI-6 predictive model or algorithm 118, in order to rapidly and accurately evaluate patient trauma. In some examples, NEI-6 predictive model 118 may include, as non-limiting examples, logistic regression models and/or random-forest models, configured to construct a pair of predictive algorithms based on clinically relevant patient-parameter variables included in patient data 104 to predict both ISS and NEI-6. In some such examples, restricted cubic splines may be used to model nonlinear determinative factors.

As detailed further below, the accuracy of the resulting NEI-6 predictive model(s) 118 may be assessed in terms of discriminative performance. In some implementations, the outputs 122 of NEI-6 predictive model 118 may be used in in conjunction with a tiered trauma-team-activation (TTA) system in order to help allocate available medical resources in a manner proportional to the needs (e.g., the injury burden) of patient 102.

The following descriptive example illustrates one non-limiting technique for developing, training, and evaluating a machine-learning-based NEI-6 predictive model 118. For instance, a regional trauma-quality collaborative may be used to identify and retrieve a set of trauma-patient data for all trauma patients above a certain age, such as above about 16 years old, from Level 1 and Level 2 trauma centers verified by the American College of Surgeons' Committee on Trauma (ACS-COT). The retrieved patient data may be divided into a “training” (or “derivation”) dataset and a “validation” (or “evaluation”) dataset.

The Michigan Trauma Quality Improvement Program (MTQIP) is an example of such a collaborative quality initiative composed of thirty-one ACS-COT-verified Level-1 and Level-2 trauma centers in Michigan and Minnesota. MTQIP utilizes a data-definitions dictionary based upon the National Trauma Data Standard (NTDS), which is published online and updated annually. Trauma-data abstractors from participating hospitals undergo training in MTQIP and NTDS data definitions. Data may be transmitted from the trauma registry at participating hospitals to the coordinating center according to two-month intervals. The MTQIP database, instead of the National Trauma Data Bank (NTDB) or the American College of Surgeons Trauma Quality Improvement Program (ACS-TQIP), may be used in some examples, since the MTQIP includes patient TTA level. An example of inclusion criteria applied to form a MTQIP patient cohort is as follows: age≥16 years old; ISS≥5; primary mechanism of injury classified as either “blunt” or “penetrating.” “Blunt” may be defined as “an injury where the primary ICD-9 External Cause Code (“E-code”) is mapped to the following categories: fall, machinery, motor vehicle traffic, pedestrian, cyclist, and struck by/against a blunt object (assault).” “Penetrating” may be defined as “an injury where the primary E-code is mapped to the following categories: cut/pierce, and firearm.” ISS values may be derived from registrar-abstracted-and-recorded Abbreviated Injury Scale (AIS) 2005 codes with AIS 2008 updates. Patients directly admitted (e.g., without EMS intervention or evaluation), and/or patients admitted with no signs of life, may be excluded from the dataset.

In a particular example of the training and validation datasets, a cohort of 22,069 patients may be determined to satisfy a set of inclusion criteria, as described above. Of these, 12,624 patients may be included in the training dataset, and 9,445 patients may be included in the internal prospective validation dataset. Based on the training dataset of 12,624 patients (e.g., 62.6% male; median age 61 years; median ISS 9) and the validation dataset from 9,445 patients (e.g., 62.6% male; median age 59 years; median ISS 9), the following six significant predictive parameters (e.g., patient data 104) may be selected as a basis for developing an NEI-6 predictive model 118: (1) age, (2) gender, (3) field Glasgow coma score (GCS), (4) vital signs, (5) intentionality (e.g., accidental vs. intentional injury), and (6) mechanism of injury. In other examples, more, fewer, or different predictive parameters may be selected.

In the training data set, 62.6% of the patients may be male (e.g., median [IQR] age of 61 [38-80] years old; median ISS of 9 [8-14]). In the validation data set 62.6% may be male (e.g., median [IQR] age of 59 [36-78] years old; median ISS of 9 [6-14]). In some such examples, NEI-6-positive patients may be more likely to be older, to be male, and to die from their injuries in both the training and the validation cohorts, as compared to NEI-6-negative patients (See Table 2, below). In some examples, all of the indicated injury types may be significantly different for NEI-6-positive patients compared to those for NEI-6-negative patients, thereby illustrating the overall-higher burden of traumatic illness for patients needing an emergent intervention. Patients in the validation cohort and the training cohort may be similar overall in terms of demographics, physiologic and injuries. For both the training and the validation cohorts, “blunt force” may be the most common mechanism of injury (e.g., greater than about 98% of indicated injuries, see Table 2, below).

From within the training dataset, each patient's NEI-6 designation (NEI-6 Positive or NEI-6 Negative) may be selected and extracted as the outcome of interest. As will be readily understood by those of skill in the art, an “NEI-6” designation for a patient 102 depends on the performance of any one or more of the following six interventional procedures on a patient: (1) receiving ≥5 units of packed red blood cells within the first 4 hours, (2) any operation, (3) an angiography, (4) a chest tube placement, (5) a central line placement, or (6) a brain intervention (e.g., placement of an intracranial pressure monitor, craniotomy, etc.) within 6 hours of patient arrival to the emergency department (ED). More specifically, patients who have undergone any of these six interventions within the first 6 hours of hospital arrival are categorized as “NEI-6 positive,” whereas patients who have not received any of these six procedures are categorized as “NEI-6 negative.”

In some examples, a plurality of input parameters may be selected, based on available clinical knowledge and prior literature, for determining whether a patient should be categorized as NEI-6 positive or NEI-6 negative. These patient parameters may include, in one illustrative example: age, gender, firearm injury, insurance, legal intervention, penetrating injury, fall injury, unintentional injury, central gunshot wound, field GCS, field SBP, field pulse, ED temperature, obesity, transport time<15 minutes, transport time<30 minutes, life-support-during-transport method (e.g., advanced life support vs basic life support), transfer-origin-location type (scene vs. transfer from another hospital) and evening arrival.

For development of NEI-6 predictive model 118, some examples of feature-selection methods, which may be used to narrow-down a set of potential input variables to those most-useful for the predictive model 118, may include all features; generalized local learning; recursive feature elimination. Some example classifiers include: logistic regression; random forest; boosted tree; and support vector machines (SVMs). In particular, logistic regression may be selected for the ability of this technique to provide a reason or justification for the determined NEI-6 designation it generates. Specifically, an NEI-6 predictive model 118 that includes a logistic-regression-based model can be configured to provide an indication of a basis for its selected NEI-6 designation based on determined coefficients of the patient parameters provided within patient data 104. In some instances, providing an indication of a basis for the NEI-6 determination in this way can help fulfill and/or reduce mandatory approval requirements by a relevant regulatory authority.

In some examples, a hyperparameter (e.g., a value used to control the training process) for random forest may include 500 trees and the square root of a number of variables for the number of variables available for splitting (e.g., “mtry”). For example, a hyperparameter for boosted tree using a “tree stump” may include 500 trees and a learning rate of 0.1. For some (but not all) examples implementing SVMs, the Cartesian product of the following hyperparameters: degree=[1, 2, 3, 4], and C=[0.01, 0.1, 1, 10, 100]. In some cases, these hyperparameters may result in 69 combinations of feature selection, and classification method.

To select the optimal number of input variables, hyperparameter combinations may be used for this task. For instance, a stratified repeated nested cross-validation may be used to achieve optimal model selection, avoid overfitting, and obtain unbiased performance estimation. The selection of the optimal combination of feature selection methods, model types and hyperparameters may be conducted in the inner loop of the nested cross-validation, and the “best” model may be determined by the best average inner-loop model performance. Categorical variables may include converted multiple binary variables with one-hot encoding. Imputation may be used to assign a default value for a missing parameter. For example, missing data may be handled by median imputation and including indicator variables for missing patient parameters. In other words, for cases in which a variable was not available, the average value (or median or weighted average in other examples) for that variable within the training data may be “filled in.” Imputed variables may include, as non-limiting examples: field SBP, pulse, GCS, and temperature (See Table 1 below, showing examples of percentages of missing values for each patient-parameter variable). This imputation may be performed based on the training data at all stages of the model development and performance estimation, in order to prevent information leakage.

TABLE 1
Variables with missing values in the datasets and
the percent of values missing for each variable.

| Variable | % Missing |
|---|---|
| Temperature | 12.64 |
| Field SBP | 3.18 |
| Field Pulse | 2.21 |
| Field GCS | 3.64 |

In some examples, the resulting predictive model 118 includes three demographic-type variables [e.g., (1) age, (2) gender, and (3) insurance]; six mechanistic variables [e.g., (1) firearm injury, (2) legal intervention, (3) penetrating injury, (4) fall-type injury, (5) unintentional injury, and (6) central GSW]; four physiologic variables [e.g., (1) field GCS, (2) field SBP, (3) field pulse, and (4) ED temperature]; and three transport-type variables [e.g., (1) transport time less than 15 minutes, (2) transport time less than 30 minutes, and (3) evening arrival]. Accordingly, an NEI-6 trauma-triage predictive model 118 as described herein may use prehospital metrics (e.g., patient data 104) to determine a patient's need for a highest-available level of trauma activation 124. In this way, a prehospital evaluation of major trauma can help reduce undertriage-based mortality and improve allocation of limited medical resources.

Once a suitable predictive model 118 is developed and evaluated, the model 118 may be implemented as software, such as a publicly available website and/or mobile application, for use as a convenient and quick trauma-hospital-activation tool, e.g., to determine an appropriate level of trauma activation 124 in the prehospital setting. In such examples, the website and/or mobile application may be used to rapidly provide trauma-activation-level recommendations, e.g., by evaluating the boosted-stump machine-learning model on input data 104 provided by a user. In one example implementation, a software architecture can include a RESTful API that accepts NEI-6 determination requests containing validated and formatted input. The API can be used to compute an NEI-6 designation determination 122 via a MATLAB engine, in which the machine-learning model 118 runs using the Java Engine API. The RESTful API then returns the NEI-6 determination 122 and a corresponding trauma-level-activation recommendation 124 to the user via a user interface, such as display 108.

TABLE 2
Demographics for the training (2016-2017 data) and validation (2018 data)

| | Training Cohort | | | | | Validation Cohort | | | | |
|---|---|---|---|---|---|---|---|---|---|---|
| | NEI-6 Neg. | | NEI-6 Pos. | | P- | NEI-6 Neg. | | NEI-6 Pos. | | P- |
| Variable | (N = 11069) | | (N = 1555) | | value | (N = 8364) | | (N = 1081) | | value |
| Age (years) | 61.6 | (±22.9) | 49.5 | (±20.9) | <0.001 | 64.0 | (±21.9) | 49.9 | (±21.3) | <0.001 |
| Mean (±SD) | | | | | | | | | | |
| Sex (%) | | | | | | | | | | |
| Male | 5489 | (49.6) | 418 | (26.9) | <0.001 | 4288 | (51.3) | 309 | (28.6) | <0.001 |
| Female | 5580 | (50.4) | 1137 | (73.1) | | 4076 | (48.7) | 772 | (71.4) | |
| Race (%) | | | | | | | | | | |
| Black | 1823 | (16.5) | 400 | (25.7) | <0.001 | 1318 | (15.8) | 285 | (26.4) | <0.001 |
| Other | 490 | (4.4) | 77 | (5.0) | | 281 | (3.4) | 57 | (5.3) | |
| White | 8756 | (79.1) | 1078 | (69.3) | | 6765 | (80.9) | 739 | (68.4) | |
| Days in hospital | | | | | | | | | | |
| Mean (±SD) | 4.4 | (±4.34) | 11.0 | (±12.9) | <0.001 | 4.5 | (±4.8) | 9.4 | (±9.3) | <0.001 |
| Median | 3.0 | | 7.0 | | | 4.0 | | 7.0 | | |
| Missing (%) | 12 | (0.1) | 33 | (2.1) | | 2 | (0.02) | 18 | (1.7) | |
| Mortality (%) | | | | | | | | | | |
| Survivor | 10825 | (97.8) | 1140 | (73.3) | <0.001 | 8231 | (98.4) | 811 | (75.0) | <0.001 |
| Non-survivor | 244 | (2.2) | 415 | (26.7) | | 133 | (1.6) | 270 | (25.0) | |
| Private Insurance | | | | | | | | | | |
| (%) | | | | | | | | | | |
| Yes | 4187 | (37.8) | 744 | (47.8) | <0.001 | 2933 | (35.1) | 509 | (47.1) | <0.001 |
| No | 6882 | (62.2) | 811 | (52.2) | | 5431 | (64.9) | 572 | (52.9) | |
| Evening arrival | | | | | | | | | | |
| (%) | | | | | | | | | | |
| Yes | 3658 | (33.0) | 659 | (42.4) | <0.001 | 2727 | (32.6) | 443 | (41.) | <0.001 |
| No | 7411 | (67.0) | 896 | (57.6) | | 5637 | (67.4) | 638 | (59.0) | |
| Open fracture | | | | | | | | | | |
| (%) | | | | | | | | | | |
| Yes | 355 | (3.2) | 91 | (5.9) | <0.001 | 254 | (3.0) | 60 | (5.6) | <0.001 |
| No | 10714 | (96.8) | 1464 | (94.1) | | 8110 | (97) | 1021 | (94.4) | |
| Central GSW (%) | | | | | | | | | | |
| Yes | 61 | (0.6) | 126 | (8.1) | <0.001 | 38 | (0.5) | 100 | (9.3) | <0.001 |
| No | 11008 | (99.4) | 1429 | (91.9) | | 8326 | (99.5) | 981 | (90.7) | |
| Transfer (%) | | | | | | | | | | |
| Yes | 997 | (9.0) | 162 | (10.4) | <0.001 | 618 | (7.4) | 113 | (10.5) | <0.001 |
| No | 10072 | (91.0) | 1393 | (89.6) | | 7746 | (92.6) | 968 | (89.5) | |
| Obesity (%) | | | | | | | | | | |
| Yes | 494 | (4.5) | 57 | (3.7) | 0.32 | 320 | (3.8) | 42 | (3.9) | 0.32 |
| No | 10575 | (95.5) | 1498 | (96.3) | | 8044 | (96.2) | 1039 | (96.1) | |
| Helmet Use (%) | | | | | | | | | | |
| Yes | 433 | (3.9) | 76 | (4.9) | 0.003 | 328 | (3.9) | 59 | (5.5) | 0.004 |
| No | 10636 | (96.1) | 1479 | (95.1) | | 8036 | (96.1) | 1022 | (94.5) | |
| GCS | | | | | | | | | | |
| Mean (±SD) | 14.6 | (±1.36) | 10.6 | (±4.89) | <0.001 | 14.6 | (±1.28) | 10.8 | (±4.85) | <0.001 |
| Missing (%) | 416 | (3.8) | 44 | (2.8) | | 420 | (5.0) | 58 | (5.4) | |
| SBP | | | | | | | | | | |
| Mean (±SD) | 145 | (±28.6) | 129 | (±39.7) | <0.001 | 146 | (±28.8) | 131 | (±38.8) | <0.001 |
| Missing (%) | 260 | (2.3) | 141 | (9.1) | | 147 | (1.8) | 105 | (9.7) | |
| Pulse | | | | | | | | | | |
| Mean (±SD) | 86.9 | (±18.4) | 92.4 | (±28.0) | <0.001 | 86.9 | (±18.5) | 92.4 | (±29.2) | <0.001 |
| Missing (%) | 229 | (2.1) | 50 | (3.2) | | 106 | (1.3) | 40 | (3.7) | |
| ED Temperature | | | | | | | | | | |
| Mean (± SD) | 98.0 | (±1.05) | 97.4 | (±1.62) | <0.001 | 98.0 | (±0.820) | 97.5 | (±1.67) | <0.001 |
| Missing (%) | 1089 | (9.8) | 507 | (32.6) | | 831 | (9.9) | 312 | (28.9) | |
| Mechanism of | | | | | | | | | | |
| Injury (%) | | | | | | | | | | |
| Cut | 121 | (1.1) | 78 | (5.0) | <0.001 | 81 | (1.0) | 61 | (5.6) | <0.001 |
| Fall | 6380 | (57.6) | 460 | (29.6) | | 5230 | (62.5) | 329 | (30.4) | |
| Firearm | 200 | (1.8) | 200 | (12.9) | | 136 | (1.6) | 149 | (13.8) | |
| MCC | 491 | (4.4) | 118 | (7.6) | | 384 | (4.6) | 89 | (8.2) | |
| MVC | 2544 | (23.0) | 415 | (26.7) | | 1591 | (19.0) | 258 | (23.9) | |
| Other | 302 | (2.7) | 74 | (4.8) | | 235 | (2.8) | 71 | (6.6) | |
| Pedal | 584 | (5.3) | 152 | (9.8) | | 359 | (4.3) | 81 | (7.5) | |
| Struck | 447 | (4.0) | 58 | (3.7) | | 348 | (4.2) | 43 | (4.0) | |
| Intentionality (%) | | | | | | | | | | |
| Legal | 0 | (0) | 1 | (0.1) | <0.001 | 0 | (0) | 0 | (0) | <0.001 |
| intervention/war) | | | | | | | | | | |
| Assault | 237 | (2.1) | 197 | (12.7) | | 157 | (1.9) | 151 | (14.0) | |
| Self-Inflicted | 56 | (0.5) | 77 | (5.0) | | 41 | (0.5) | 44 | (4.1) | |
| Undetermined | 419 | (3.8) | 85 | (5.5) | | 300 | (3.6) | 73 | (6.8) | |
| Unintentional | 10357 | (93.6) | 1195 | (76.8) | | 7866 | (94.0) | 813 | (75.2) | |
| Transport dist. | | | | | | | | | | |
| (%) | | | | | | | | | | |
| >30 min | 2660 | (24.0) | 262 | (16.8) | <0.001 | 1788 | (21.4) | 198 | (18.3) | <0.001 |
| 15-30 min | 4549 | (41.1) | 445 | (28.6) | | 3577 | (42.8) | 300 | (27.8) | |
| Less than 15 min | 3860 | (34.9) | 848 | (54.5) | | 2999 | (35.9) | 583 | (53.9) | |

cohorts separated by NEI-6-Positive (Needs Emergent Intervention within 6 hours) and NEI-6-Negative (does not Need Emergent Intervention within 6 hours) classifications of patients.

As described above, current trauma triage guidelines have been shown to be insensitive in determining (or “predicting”) critical medical resource use. As used herein, “critical resource use” is defined the occurrence of any of the following within 24 hours of ED arrival: (1) emergent airway intervention in the ED, (2) major non-orthopedic surgical intervention, (3) interventional radiology procedures, (4) blood transfusion≥6 units (or any blood transfusion in a child), or (6) death (similar to NEI-6 criteria). Consequently, current hospital-triage guidelines are suboptimal in assessing the need for high resource utilization. The NEI-6 predictive models 118 described herein can accurately determine both ISS>15 and NEI-6 designation 122, and consequently, could potentially lower undertriage rates by using a more suitable metric for identifying severely injured patients 102 and recommending an appropriate level of trauma activation 124. In addition to using a superior (e.g., more-nuanced) definition of “major trauma” than just the ISS, the present NEI-6 models 118 may incorporate vital signs, GCS, and age as continuous variables, and add other variables such as “intentionality” and “central gunshot wound (GSW).” Furthermore, the present models 118 may assign different weights to each variable depending on its relative ability (e.g., influence) to classify patients as “NEI-6 Positive” or “NEI-6 Negative.”

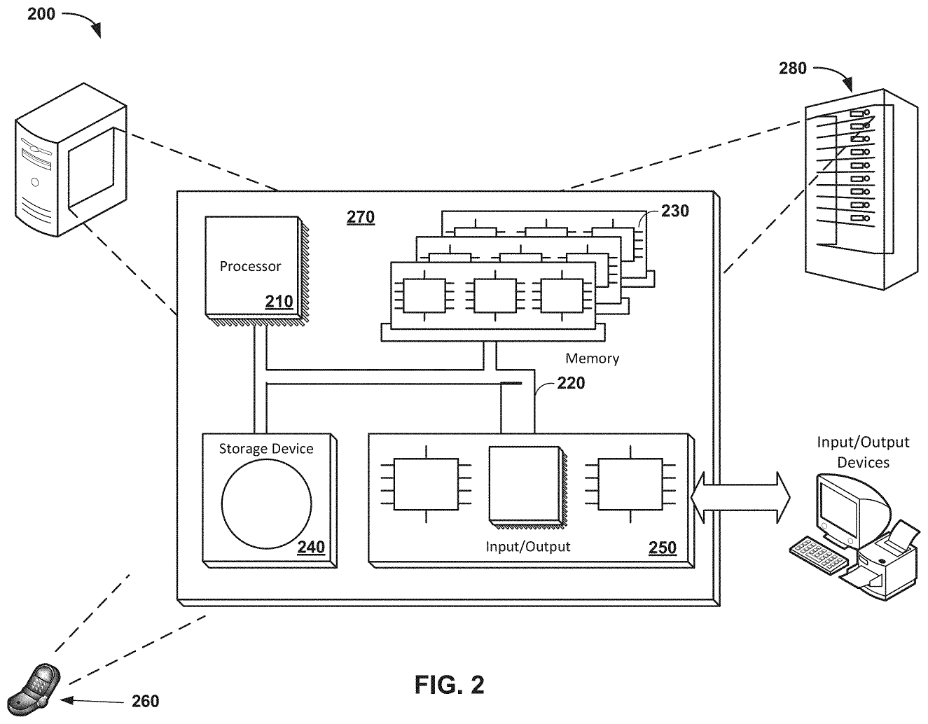

FIG. 2 is a block diagram illustrating a detailed example of various devices that may be configured to implement one or more techniques of the present disclosure. That is, computing system 200 of FIG. 2 provides an example implementation of computing system 108 of FIG. 1 for determining an NEI-6 designation 122 for a patient 104. Computing system 200 may include one or more of a mobile device (e.g., a smartphone, laptop, tablet, a personal digital assistant [PDA], or other mobile device) 260, a workstation, a computing center, a cluster of servers 280, and/or other examples of a computing environment 200, centrally located or distributed, that is capable of executing the techniques described herein. Any or all of the devices may, for example, implement portions of the techniques described herein for determining an NEI-6 designation (e.g., NEI-6 Positive or NEI-6 Negative) 122 for trauma patients 102. In some examples, functionality of NEI-6 determination system 100 may be distributed across multiple computing devices, such as a cloud-based computing system for computing the determined NEI-6 designations 122 and generating corresponding reports, and a client device, such as a tablet or mobile phone, for accessing and viewing the reports.

In the example of FIG. 2, computing device 270 includes a processor 210 that is operable to execute program instructions or software, causing the computer to perform various methods or tasks, such as performing the techniques for generating and/or using multiparametric models for NEI-6 determination as described herein. Processor 210 is coupled via bus 220 to a memory 230, which is used to store information such as program instructions and/or other data while the computer is in operation. A storage device 240, such as a hard disk drive, nonvolatile memory, or other non-transient storage device stores information such as program instructions, data files of the multidimensional data and the reduced data set, and other information. The computer also includes various input-output elements 250, including parallel or serial ports, USB, Firewire or IEEE 1394, Ethernet, and other such ports to connect the computer to external devices such a printer, video camera, display device, medical imaging device, surveillance equipment, or the like. Other input-output elements include wireless communication interfaces such as Bluetooth, Wi-Fi, and cellular data networks.

Computing device 270 may itself be a traditional personal computer, a rack-mount or business computer or server, or any other type of computerized system. Computing device 270, in a further example, may include fewer than all elements listed above, such as a thin client or mobile device having only some of the shown elements. In another example, computing device 270 is distributed among multiple computer systems, such as a distributed server that has many computers working together to provide various functions.

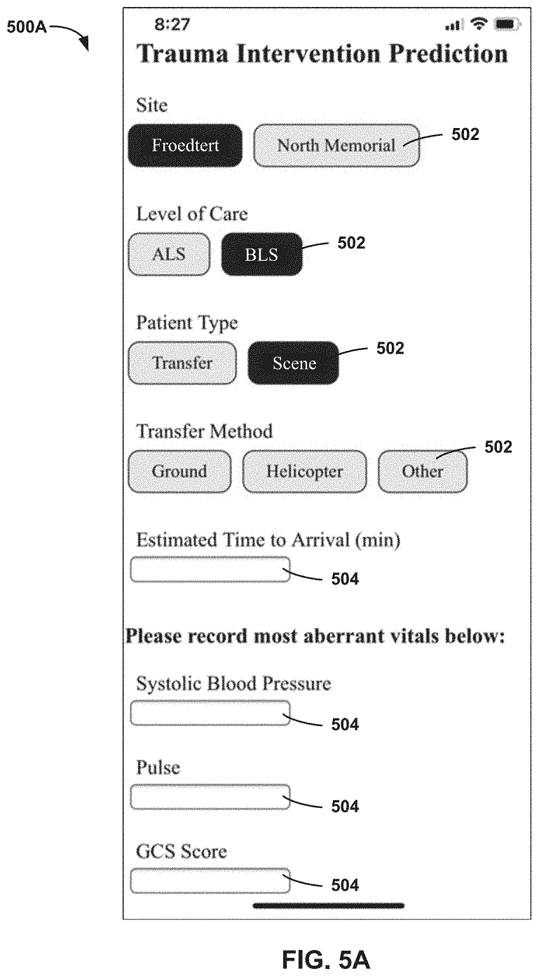

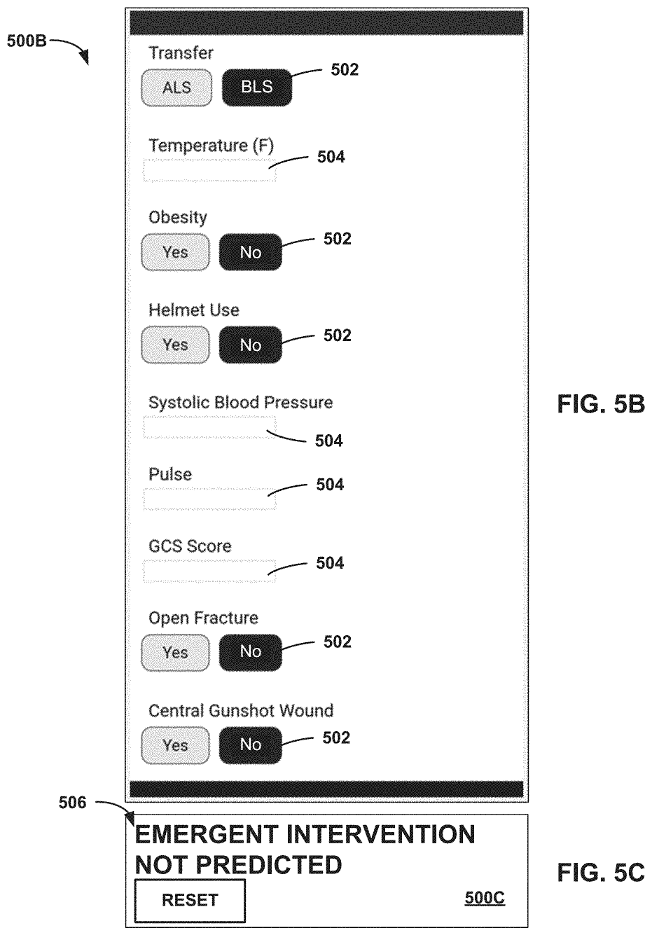

In one non-limiting example, computing system 200 includes a mobile computing device 260 and a remote server 280 configured to collectively execute the functionality of computing system 108 of FIG. 1. For instance, a user (e.g., EMS personnel) may submit user data 104 via a user interface (e.g., FIGS. 5A and 5B) presented on display 106 of mobile device 260. In other words, processing circuitry of mobile device 260 may control display 106 to present, or display, the user interface. User data 104 may be received by the processing circuitry of mobile device 260 and via the user interface. In some examples, the display 106 may be a presence-sensitive screen (e.g., a touch screen) configured to receive user input selecting or inputting user data 104 to one or more fields of the user interface. Mobile device 260 may then transmit the user data 104 (including the plurality of patient parameters) to remote server 280. Remote server 280 is configured to execute NEI-6 predictive model 118 to determine NEI-6 designation 122, as described above, and in some examples, a corresponding recommended triage activation 124. Remote server 280 may then transmit NEI-6 designation 122 and/or recommended triage activation 124 back to mobile device 260 for display via the GUI on display 106.

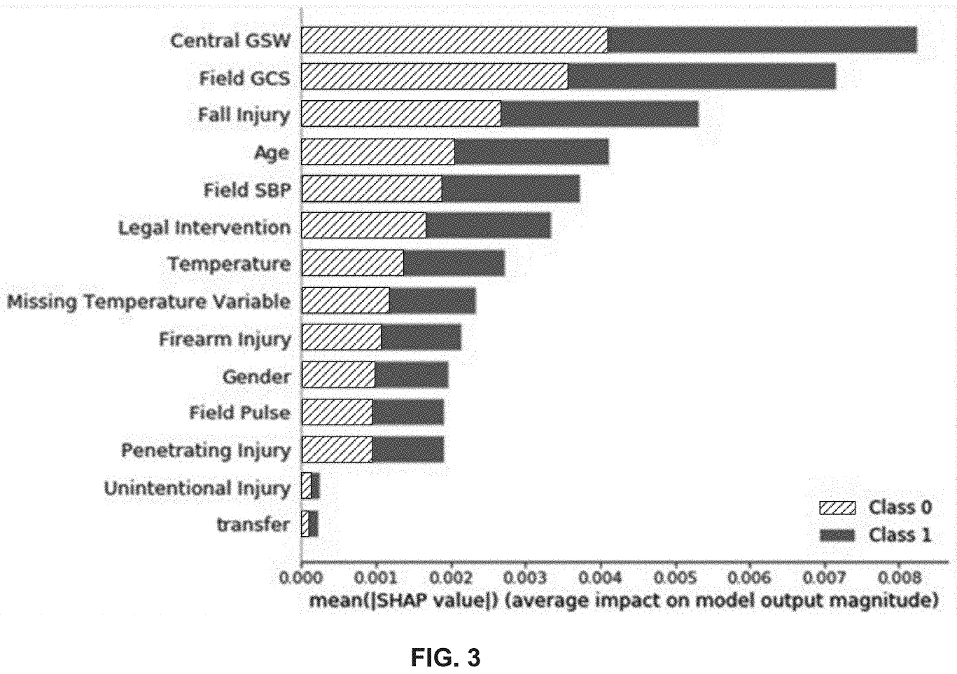

FIG. 3 is a bar graph illustrating Shapley values for an example validation dataset that may be used to evaluate the predictive models described herein. The Shapley values indicate a relative weight or “predictive importance” exhibited by each input variable 104 in the model 118 while classifying samples. The absence of a patient parameter from the bar graph of FIG. 3 indicates that the parameter had no influence in the predictive model. As shown in the specific example of FIG. 3, the top-three predictive variables (e.g., having the highest Shapley values) for a designation of “NEI-6 Positive” may be: (1) central gunshot wound (e.g., a GSW to thorax or abdomen), (2) field GCS, and (3) fall-type injury.

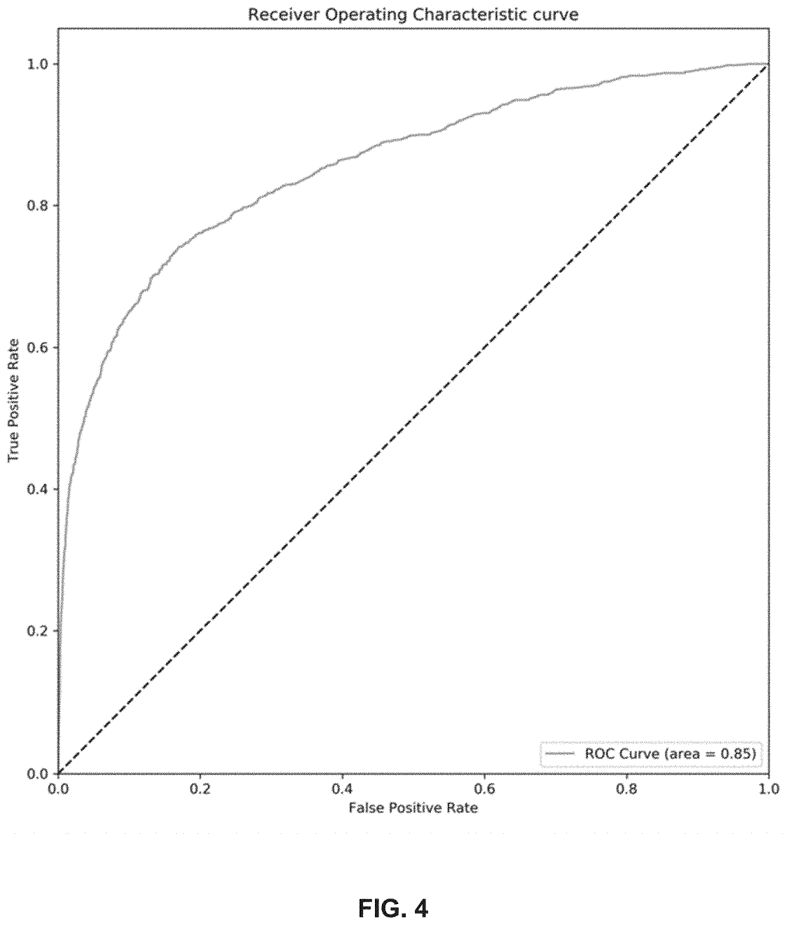

FIG. 4 is an is a line graph depicting an example Area-Under-the-Receiving-Operator-Characteristic (“AUC” or “AUROC”) curve, indicating an evaluation of a relative performance of the NEI-6 predictive models 118 of this disclosure. In some examples, NEI-6 predictive model 118 for determining a patient NEI-6 designation 122 may include a boosted-tree model, which may produce an AUC of about 0.85.

In some examples herein, a prospective evaluation (or “validation”) design may be utilized to assess the performance of NEI-6 predictive model 118. For instance, data collected during the years of 2016 and 2017 may be used to develop (e.g., “train”) an NEI-6 predictive model, and data collected during the year 2018 may be used to evaluate the predictive model. In some cases, model-validation data may be stored in a separate location, e.g., never accessed until the model-development process is completed and a final predictive model is ready for validation.

Performance estimation is used to assess the “generalizability” (e.g., applicability to data other than the training data) of a predictive model. In the present example, the performance estimation of the selected model on the model-development data may be conducted in the outer loop of the nested cross-validation. To obtain a final model, the best feature-selection methods, model types, and hyperparameters may be applied to the entire model-development data. This final model may then be applied to the model-validation data to obtain the model-performance estimation using the validation data (e.g., the 2018 data). In some examples, the “Area Under the Receiver Operating Characteristic (“AUROC” or “AUC”) curve may be used as a metric for performance estimation.

Example NEI-6 predictive models 118 developed in accordance with the techniques described herein may exhibit significant determinative performance. For instance, one AUC indication of a mean outer-loop cross-validated performance estimation on the model development dataset may be 0.85±0.02. The “best” feature-selection and classifier combination may manifest in examples using all features with a boosted-tree model. For example, a final boosted-tree model trained on the entire model development set (e.g., all trauma-patient data from the 2016-2017 “training” dataset), applied to the year-2018 model-validation dataset, can be shown to achieve an AUROC of about 0.85 in the overall cohort, and about 0.82 for a subset of the data that only includes patients of age 65 or older. These results indicate that predictive models 118 developed using available data from 2016 and 2017 retain good performance that may be generalized to the patient data from 2018.

In some examples, certain prehospital variables of patient data 104 may be excluded from consideration by the model 118, such as prehospital variables that can be recorded by EMS providers at the scene. Excluding in-hospital parameters may enhance the applicability of the present models 118 to real-world settings where not all relevant information is readily available. It is well recognized that older adults are at particular risk of undertriage. “Age” and “fall,” the most common mechanism of injury in older adults, are important determinative factors of NEI-6. In fact, the models 118 described herein provide adequate performance in the older-adult population, with an AUC of about 0.82. “Vital signs” and “GCS score” may be incorporated into the models as continuous variables. “Prehospital SBP” and “pulse” are early markers of hemodynamic instability and correlate with ED vital signs. “Hypothermia” has been shown to be an independent predictor of mortality in trauma. “GCS” shows strong association with trauma severity and is a recognized identifier of major trauma in the prehospital setting. Including “mechanism of injury” in triage models 118 increases their sensitivity and specificity. “Fall” and “firearm exposure” are among the most common causes of injury leading to death, and accordingly, both may be included in the present models 118 due to their association with major trauma and mortality. “Intentional injury” carries a higher mortality risk, as do penetrating injuries, making both variables reasonable choices for inclusion in the present model 118.

FIGS. 5A-5C depict an example user interface of a mobile application configured to implement NEI-6 predictive model 118 of FIG. 1. More specifically, FIGS. 5A-5C depict user interfaces (UIs) 500A, 500B, and 500C, respectively, such as graphical user interfaces (GUIs), generated and presented by NEI-6 predictive model 118 of FIG. 1. For example, GUIs 500A-500C may be generated by processing circuitry of computing device 270 and output for display on display 106 (FIG. 1) of computing device 270 of FIG. 2, such as a smartphone, tablet or laptop. The processing circuitry of computing device 270 may control display 106 to present any of the screens of GUIs 500A-500C.

In some examples, the mobile application may be referred to as “Trauma Intervention Prediction (TIP).” In some examples, computing device 270 may execute a mobile application that includes several different mobile applications directed to different types of predictions, and the TIP mobile application described here may be presented as one of the types of prediction applications provided by the general application. Through the user interface of the TIP application, a user, such as EMS personnel, is able to input the various prehospital variables via a mobile device, and the TIP application utilizes one or more of the predictive models 118 described above to recommend whether the trauma patient 102 requires a trauma-team activation (TTA) 124 based on the patient's likelihood 122 of being NEI-6 positive. Such examples may reduce or eliminate the need for EMS personnel to memorize complex triage rules and could potentially increase compliance with standard triage procedures. are example

FIGS. 5A and 5B depict GUIs 500A and 500B, respectively. GUIs 500A, 500B include a plurality of input buttons 502 and fields 504 enabling a user of computing system 108 to submit patient parameters according to information initially known about patient 102. As shown in GUIs 500A and 500B, example input buttons 502 may include a selectable “site” that includes a separate button for each respective hospital or health care site, a selectable “level of care” that includes a separate buttons for each available level of care transport (e.g., “ALS” (advanced life support) or “BLS” (basic life support)), a selectable “patient type” that includes separate buttons for each type of patient that was received (e.g., a patient that was transferred in “transfer” or received at the “scene” of the trauma), a selectable “transfer method” that includes separate buttons for each type of transportation method for the patient (e.g., “ground”, “helicopter”, and “other”), a selectable “obesity” field that includes buttons for “yes” and “no”, a selectable “Helmet Use” field that includes buttons for “yes” and “no”, a selectable “open fracture” field that includes buttons for “yes” and “no”, and a selectable “central gunshot wound” field that includes buttons for “yes” and “no.” These are just some example input buttons 502. Instead of separate buttons for each selectable option of buttons 502, GUIs 500A and 500B may include one or more drop down menus, text entry fields, dials, sliders, or any combination thereof.

Example fields 504 include fields such as “estimated time to arrival (min),” “systolic blood pressure,” “pulse” (pulse rate or heart rate), “GCS Score” (Glasgow Coma Scale), and “temperature (F)”. These and/or other fields may be included in fields 504. In some examples, GUIs 500A and 500B are provided together for accepting input from the user. Instead of separate text fields for each selectable option for fields 504, GUIs 500A and 500B may include one or more drop down menus, text entry fields, dials, sliders, or any combination thereof. In other examples, each of GUIs 500A and 500B may be separate and used to accept different types of inputs, where the inputs are selected for each GUI based on the location of the patient, type of responder, or any other information. In some examples, computing device 270 may generate the inputs of the GUI based on already received information related to the patient or determining the score, such that only those inputs still needed from the user are presented. Although two screens are shown as including all of input buttons 502 and input fields 504, GUI 500A and 500B may instead include a single screen or three or more screens with respective inputs and/or scrollable screens that enable GUIs 500A and 500B to provide additional inputs that may not fit or be shown at the same time on display 106.

After selecting one or more input buttons 502, as well as inputting the other available patient parameters into input boxes 504, the TIPs application may generate GUI 500C depicted in FIG. 5C. GUI 500C includes an indication 506 of a determined NEI-6 designation 122 for patient 102. For instance, in the example shown in FIG. 5C, GUI 500C displays an indication 506 that patient 102 is determined to be NEI-6 Negative (e.g., “emergent intervention not predicted”).

In some examples of a software application implementing NEI-6 predictive models 118 as described herein, an initial GUI display (not shown in FIGS. 5A-5C) may include a dropdown menu or other user-input mechanism prompting a user to select an appropriate or desired predictive model from among a plurality of different types of predictive models. For instance, the GUI may prompt the user to select between an NEI-6 predictive model (as described herein), an “ISS>15” predictive model, an Elderly Mortality After Trauma (EMAT) predictive model, or another similar trauma-related software application.

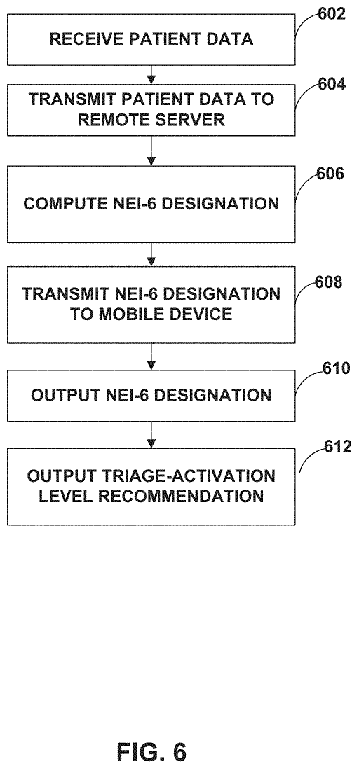

FIG. 6 is a flowchart illustrating an example operation in accordance with the techniques of this disclosure. As shown in FIG. 1, computing system 108 (e.g., a mobile computing device 260) receives initial patient parameters 104 (602). In some examples, the mobile computing device 260 may receive user input via a user interface and then transfer the patient parameters 104 to a remote server 280 of computing system 200, where the patient parameters 104 may be stored into data repository 116.

Based on initial patient data 104, remote server 280 executes NEI-6 predictive model 118 which determines whether patient 102 is likely to be designated as NEI-6 Positive or NEI-6 Negative (606). For example, NEI-6 predictive model 118 may be configured to determine a patient NEI-6 designation 122 by submitting patient parameters 104 to one or more machine-learning models or algorithms trained on historical trauma-patient data to determine a subsequent NEI-6 designation for patient 102. Therefore, remote server 280 may receive the output from NEI-6 predictive model 118 that indicates the patient NEI-designation 122.

After determining an NEI-6 designation 122 for patient 102, remote server 280 may transfer the NEI-6 designation 122 back to mobile computing device 260 (608), which may then output the NEI-6 designation for display via a GUI (610). In some examples, but not all examples, mobile computing device 260 may additionally output for display (or control the display to output) a corresponding triage-activation-level recommendation 124 associated with the patient NEI-6 designation 122 (612).

The following examples are described herein:

Example 1: A computing system includes processing circuitry configured to receive, from a mobile computing device, user input comprising a plurality of parameters indicating a condition of a trauma patient; apply the plurality of parameters to one or more machine-learning algorithms trained to determine, based on the plurality of parameters, a trauma-triage category for the patient, wherein the trauma-triage category for the patient indicates a trauma-triage recommendation for the patient; and transmit the trauma-triage category to the mobile computing device for display on the mobile computing device.

Example 2: The computing system of example 1, wherein the trauma-triage category further indicates a Need for Emergent Intervention within 6 hours (NEI-6) designation for the patient.

Example 3: The computing system of example 2, wherein, to determine the trauma-triage category for the patient, the processing circuitry is configured to select the NEI-6 designation for the patient from a group comprising NEI-6 Positive and NEI-6 Negative.

Example 4: The computing system of example 3, wherein an NEI-6 Positive designation for the patient indicates that the patient is in need of at least one of the following procedures within 6 hours of an arrival of the patient to an emergency department (ED): receiving 5 or more units of packed red blood cells within four hours of the arrival to the ED; any surgical operation; an angiography; a chest-tube placement; a central-line placement; or a brain-intervention procedure.

Example 5: The computing system of any of examples 1-4, wherein the trauma-triage recommendation indicates recommended levels or types of trauma treatment for the patient.

Example 6: The computing system of any of examples 1-5, wherein the plurality of parameters comprise at least: an age of the patient; a gender of the patient; a field Glasgow Coma Scale (GCS) score of the patient; vital signs of the patient; an intentionality of the patient; and a mechanism of an injury of the patient.

Example 7: The computing system of any of examples 1-6, wherein the plurality of parameters comprise at least: an age of the patient; a gender of the patient; an obesity of the patient; a firearm injury; an insurance status of the patient; a legal intervention; a penetrating injury; a fall injury; an unintentional injury; a central gunshot wound (GSW); a field Glasgow Coma Scale (GCS) score of the patient; a field Systolic Blood Pressure (SBP) of the patient; a field pulse of the patient; an emergency department (ED) temperature of the patient; a life-support-during-transport level; a transport-origin-location type; a transport time to the ED of less than fifteen minutes; a transport time to the ED of less than thirty minutes; and an evening arrival to the ED.

Example 8: The computing system of any of examples 1-8, wherein the one or more machine-learning models comprise one of a logistic-regression model, a boosted-tree model, a boosted-stump model, or a random-forest model.

Example 9: The computing system of example 8, wherein the one or more machine-learning models comprise the logistic-regression model, and wherein the processing circuitry is further configured to generate and output a basis for the trauma-triage category determined by the logistic-regression model.

Example 10: The computing system of example 9, wherein, to generate the basis for the trauma-triage category, the processing circuitry is configured to output an indication of a set of coefficients associated with respective parameters of the plurality of parameters.

Example 11: The computing system of any of examples 1-10, wherein, the one or more machine-learning models comprise restricted cubic splines that model nonlinear determinative factors among the plurality of parameters.

Example 12: The computing system of any of examples 1-11, wherein the processing circuitry is further configured to impute a default value for a missing parameter among the plurality of parameters.

Example 13: The computing system of example 10, wherein the default value comprises an average value of the missing parameter calculated from a set of training data used to train the one or more machine-learning models.

Example 14: A computing device includes processing circuitry configured to: receive, via a user interface, user input comprising a plurality of parameters indicating a condition of a trauma patient; transmit the plurality of parameters to a remote server configured to apply the plurality of parameters to one or more machine-learning algorithms trained to determine, based on the plurality of parameters, a trauma-triage category for the patient, wherein the trauma-triage category for the patient indicates a trauma-triage recommendation for the patient; receive the trauma-triage category for the patient from the remote server; and output the trauma-triage category for the patient for display via the user interface.

Example 15: The computing device of example 14, wherein the trauma-triage category further indicates a Need for Emergent Intervention within 6 hours (NEI-6) designation for the patient.

Example 16: The computing system of example 15, wherein, to determine the trauma-triage category for the patient, the remote server is configured to select the NEI-6 designation for the patient from a group comprising NEI-6-Positive and NEI-6-Negative.

Example 17: The computing system of example 16, wherein an NEI-6-Positive designation for the patient indicates that the patient is in need of at least one of the following procedures within 6 hours of an arrival of the patient to an emergency department (ED): receiving 5 or more units of packed red blood cells within four hours of the arrival to the ED; any surgical operation; an angiography; a chest-tube placement; a central-line placement; or a brain-intervention procedure.

Example 18: The computing system of example 16, wherein, in response to receiving an NEI-6-Positive designation for the patient from the remote server, the processing circuitry is further configured to output, for display via the user interface, a recommendation for a full Trauma-Team Activation (TTA) for the patient.

Example 19: The computing system of any of examples 14-18, wherein the plurality of parameters comprises at least: an age of the patient; a gender of the patient; a field Glasgow Coma Scale (GCS) score of the patient; vital signs of the patient; an intentionality of the patient; and a mechanism of an injury of the patient.

Example 20: The computing system of any of examples 14-19, wherein the plurality of parameters comprises at least: an age of the patient; a gender of the patient; an obesity of the patient; a firearm injury; an insurance status of the patient; a legal intervention; a penetrating injury; a fall injury; an unintentional injury; a central gunshot wound (GSW); a field Glasgow Coma Scale (GCS) score of the patient; a field Systolic Blood Pressure (SBP) of the patient; a field pulse of the patient; an emergency department (ED) temperature of the patient; a life-support-during-transport level; a transport-origin-location type; a transport time to the ED of less than fifteen minutes; a transport time to the ED of less than thirty minutes; and an evening arrival to the ED.

Example 21: A method includes receiving, by first processing circuitry of a mobile computing device via a user interface of the mobile computing device, user input comprising a plurality of parameters indicating a condition of a trauma patient; wirelessly transmitting, by the first processing circuitry, the plurality of parameters to a remote server; receiving, by second processing circuitry of the remote server, the plurality of parameters; applying, by the second processing circuitry, the plurality of parameters to one or more machine-learning algorithms trained to determine, based on the plurality of parameters, a trauma-triage category for the patient, wherein the trauma-triage category for the patient indicates a trauma-triage recommendation for the patient; wirelessly transmitting, by the second processing circuitry to the mobile computing device, the trauma-triage category for the patient; receiving, by the first processing circuitry from the remote server, the trauma-triage category; and outputting for display, by the first processing circuitry via the user interface, an indication of the trauma-triage category.

Example 22: The method of example 21, wherein the trauma-triage category further indicates a comprises a Need for Emergent Intervention within 6 hours (NEI-6) designation for the patient.

Example 23. A method as described in the specification.

Example 24. A non-transitory, computer-readable medium storing instructions that, when executed by processing circuitry, cause the processing circuitry to perform one or more of the techniques described in the specification.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media, which includes any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable storage medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

What is claimed is:

1. A computing system comprising:
processing circuitry configured to:
receive, from a mobile computing device, user input comprising a plurality of parameters indicating a condition of a trauma patient;
apply the plurality of parameters to one or more machine-learning algorithms trained to determine, based on the plurality of parameters, a trauma-triage category for the patient, wherein the trauma-triage category for the patient indicates a trauma-triage recommendation for the patient; and
transmit the trauma-triage category to the mobile computing device for display on the mobile computing device.

2. The computing system of claim 1, wherein the trauma-triage category further indicates a Need for Emergent Intervention within 6 hours (NEI-6) designation for the patient.

3. The computing system of claim 2, wherein, to determine the trauma-triage category for the patient, the processing circuitry is configured to select the NEI-6 designation for the patient from a group comprising NEI-6 Positive and NEI-6 Negative.

4. The computing system of claim 3, wherein an NEI-6 Positive designation for the patient indicates that the patient is in need of at least one of the following procedures within 6 hours of an arrival of the patient to an emergency department (ED):
receiving 5 or more units of packed red blood cells within four hours of the arrival to the ED;
any surgical operation;
an angiography;
a chest-tube placement;
a central-line placement; or
a brain-intervention procedure.

5. The computing system of claim 1, wherein the trauma-triage recommendation indicates recommended levels or types of trauma treatment for the patient.

6. The computing system of claim 1, wherein the plurality of parameters comprise at least:
an age of the patient;
a gender of the patient;
a field Glasgow Coma Scale (GCS) score of the patient;
vital signs of the patient;
an intentionality of the patient; and
a mechanism of an injury of the patient.

7. The computing system of claim 1, wherein the plurality of parameters comprise at least:
an age of the patient;
a gender of the patient;
an obesity of the patient;
a firearm injury;
an insurance status of the patient;
a legal intervention;
a penetrating injury;
a fall injury;
an unintentional injury;
a central gunshot wound (GSW);
a field Glasgow Coma Scale (GCS) score of the patient;
a field Systolic Blood Pressure (SBP) of the patient;
a field pulse of the patient;
an emergency department (ED) temperature of the patient;
a life-support-during-transport level;
a transport-origin-location type;
a transport time to the ED of less than fifteen minutes;
a transport time to the ED of less than thirty minutes; and
an evening arrival to the ED.

8. The computing system of claim 1, wherein the one or more machine-learning models comprise one of a logistic-regression model, a boosted-tree model, a boosted-stump model, or a random-forest model.

9. The computing system of claim 8, wherein the one or more machine-learning models comprise the logistic-regression model, and wherein the processing circuitry is further configured to generate and output a basis for the trauma-triage category determined by the logistic-regression model.

10. The computing system of claim 9, wherein, to generate the basis for the trauma-triage category, the processing circuitry is configured to output an indication of a set of coefficients associated with respective parameters of the plurality of parameters.

11. The computing system of claim 1, wherein, the one or more machine-learning models comprise restricted cubic splines that model nonlinear determinative factors among the plurality of parameters.

12. The computing system of claim 1, wherein the processing circuitry is further configured to impute a default value for a missing parameter among the plurality of parameters.

13. The computing system of claim 10, wherein the default value comprises an average value of the missing parameter calculated from a set of training data used to train the one or more machine-learning models.

14. A computing device comprising processing circuitry configured to:
receive, via a user interface, user input comprising a plurality of parameters indicating a condition of a trauma patient;
transmit the plurality of parameters to a remote server configured to apply the plurality of parameters to one or more machine-learning algorithms trained to determine, based on the plurality of parameters, a trauma-triage category for the patient, wherein the trauma-triage category for the patient indicates a trauma-triage recommendation for the patient;
receive the trauma-triage category for the patient from the remote server; and
output the trauma-triage category for the patient for display via the user interface.

15. The computing device of claim 14, wherein the trauma-triage category further indicates a Need for Emergent Intervention within 6 hours (NEI-6) designation for the patient.

16. The computing system of claim 15, wherein, to determine the trauma-triage category for the patient, the remote server is configured to select the NEI-6 designation for the patient from a group comprising NEI-6-Positive and NEI-6-Negative.

17. The computing system of claim 16, wherein an NEI-6-Positive designation for the patient indicates that the patient is in need of at least one of the following procedures within 6 hours of an arrival of the patient to an emergency department (ED):
receiving 5 or more units of packed red blood cells within four hours of the arrival to the ED;
any surgical operation;
an angiography;
a chest-tube placement;
a central-line placement; or
a brain-intervention procedure.

18. The computing system of claim 16, wherein, in response to receiving an NEI-6-Positive designation for the patient from the remote server, the processing circuitry is further configured to output, for display via the user interface, a recommendation for a full Trauma-Team Activation (TTA) for the patient.

19. The computing system of claim 14, wherein the plurality of parameters comprises at least:
an age of the patient;
a gender of the patient;
a field Glasgow Coma Scale (GCS) score of the patient;
vital signs of the patient;
an intentionality of the patient; and
a mechanism of an injury of the patient.

20. A method comprising:
receiving, by first processing circuitry of a mobile computing device via a user interface of the mobile computing device, user input comprising a plurality of parameters indicating a condition of a trauma patient;
wirelessly transmitting, by the first processing circuitry, the plurality of parameters to a remote server;
receiving, by second processing circuitry of the remote server, the plurality of parameters;
applying, by the second processing circuitry, the plurality of parameters to one or more machine-learning algorithms trained to determine, based on the plurality of parameters, a trauma-triage category for the patient, wherein the trauma-triage category for the patient indicates a trauma-triage recommendation for the patient;
wirelessly transmitting, by the second processing circuitry to the mobile computing device, the trauma-triage category for the patient;
receiving, by the first processing circuitry from the remote server, the trauma-triage category; and
outputting for display, by the first processing circuitry via the user interface, an indication of the trauma-triage category.
